(19) United States
(12) Patent Application Publication
(10) Pub. No.: US 2026/0186095 A1
(43) Pub. Date: Jul. 2, 2026

(54) MAGNETIC RESONANCE IMAGING SYSTEM AND COIL POSITIONING METHOD THEREFOR

(71) Applicant: GE Precision Healthcare LLC, Waukesha, WI (US)

(72) Inventors: Xuelian Lu, Beijing (CN); Kun Wang, Beijing (CN); Jiabin Yao, Beijing (CN); Cunli Zhang, Beijing (CN); Qingyu Dai, Beijing (CN); Xiwen Zheng, Beijing (CN)

(21) Appl. No.: 19/427,694

(22) Filed: Dec. 19, 2025

(30) Foreign Application Priority Data
Jan. 2, 2025 (CN) 202510006630.8

(51) Int. Cl. G01R 33/58; G01R 33/34; G01R 33/36

(52) U.S. Cl. CPC G01R 33/583; G01R 33/34084; G01R 33/3692

(57) ABSTRACT

A magnetic resonance imaging system and a coil positioning method therefor are provided. The method includes: detecting an edge position of a radio frequency coil, wherein at least a portion of the radio frequency coil is located in the maximum scan field of view of a magnetic resonance imaging system; and determining, according to the edge position, a second distance by which a table is to be moved, wherein after the table is moved by the second distance, the center position of the radio frequency coil is aligned with a scan center.

### CROSS REFERENCE TO RELATED APPLICATIONS

The present application claims priority and benefit of Chinese Patent Application No. 202510006630.8 filed on Jan. 2, 2025, which is incorporated herein by reference in its entirety.

### TECHNICAL FIELD

Embodiments of the present application relate to the technical field of medical devices, and in particular to a magnetic resonance imaging system and a coil positioning method therefor.

### BACKGROUND

Magnetic resonance (MR) imaging systems have been widely used in the field of medical diagnosis. Existing magnetic resonance imaging systems typically have a main magnet, a gradient radio frequency amplifier, a gradient coil, a transmit chain module, a radio frequency coil, a receive chain module, etc. The transmit chain module generates a radio frequency pulse signal and transmits same to a transmit/receive coil. The radio frequency coil generates a radio frequency excitation signal to excite a scanned subject to generate a magnetic resonance signal. The magnetic resonance signal is received by using the radio frequency coil, and a medical parameter image may be reconstructed according to the received magnetic resonance signal.

Currently, a radio frequency coil includes a transmit/receive coil or a receive coil, including, for example, a cavity-type body coil located within a gradient coil, and a dedicated local coil (for example, used as a receive coil) used to cover a part of a patient, such as a knee coil, a shoulder coil, a spine coil, a wrist coil, a head and neck coil, etc.

### SUMMARY OF THE INVENTION

When the above dedicated local coil is used to scan a patient, it is necessary to position a coil covering a site to be examined (or a region of interest) of the patient at the scan center of a magnet of the magnetic resonance system, so as to improve imaging quality.

Current positioning technology mainly uses a positioning light, for example, a positioning light is provided at the entrance of a magnetic resonance scan space. An operator presses a positioning light key on a control panel and moves a table by means of a table movement key on the control panel. The table is continuously adjusted until a marker on a coil corresponding to the site to be examined is aligned with the positioning light (for example, light emitted by the positioning light is irradiated at the marker). A landmark key is pressed, and the position of the table when alignment is achieved is used as a home position. An advance to scan key on the control panel is triggered, so that the table will automatically travel a preset distance and then stop automatically, and the position at which the table stops causes the site to be examined and the corresponding coil to be located at a scan center of a magnet of the magnetic resonance system.

However, the inventors have found that during actual scanning, there is usually a blanket or other covering on the patient. Thus, the marker on the coil will also be covered by the covering. This makes a positioning process more complicated, requiring manual intervention and increasing time required. For this reason, the prior art also proposes a solution, in which hardware is provided on the table, and coil positioning is performed according to relative positions of the hardware, the coil, and the table in combination with a corresponding algorithm. However, the additional hardware increases device costs.

In view of at least one of the above problems, embodiments of the present application provide a magnetic resonance imaging system and a coil positioning method therefor.

According to an aspect of the embodiments of the present application, a coil positioning method for a magnetic resonance imaging system is provided. The method comprises: detecting an edge position of a radio frequency coil, wherein at least a portion of the radio frequency coil is located in the maximum scan field of view of a magnetic resonance imaging system; and determining, according to the edge position, a second distance by which a table is to be moved, wherein after the table is moved by the second distance, the center position of the radio frequency coil is aligned with a scan center.

According to an aspect of the embodiments of the present application, a magnetic resonance imaging system is provided, the system comprises: a table; a transceiver coil; a radio frequency coil; a scanning unit; and a controller, which is configured to execute the coil positioning method described in the previous aspect.

One of the beneficial effects of the embodiments of the present application is that: A second distance by which a table is to be moved is determined by detecting an edge position of a radio frequency coil when at least a portion of the radio frequency coil is located in the maximum scan field of view of a magnetic resonance imaging system. Therefore, automatic positioning of the radio frequency coil can be achieved, and no human intervention is required, which can simplify the scanning procedure and save scanning time. In addition, since it is no longer necessary to set a laser light for positioning, hardware costs can be saved, and hazards caused by the laser light irradiating the eyes during head scanning can also be prevented.

With reference to the following description and drawings, specific implementations of the embodiments of the present application are disclosed in detail, and the way in which the principles of the embodiments of the present application can be employed are illustrated. It should be understood that the implementations of the present application are not limited in scope thereby. Within the scope of the spirit and clauses of the appended claims, the implementations of the present application comprise many changes, modifications, and equivalents.

### BRIEF DESCRIPTION OF THE DRAWINGS

The included drawings are used to provide further understanding of the embodiments of the present application, which constitute a part of the description and are used to illustrate the implementations of the present application and explain the principles of the present application together with textual description. Evidently, the drawings in the following description are merely some embodiments of the present application, and those of ordinary skill in the art may obtain other implementations according to the drawings without involving inventive effort. In the drawings:

FIG. 1 is a schematic diagram of a magnetic resonance imaging system according to embodiments of the present application;

FIG. 2 is a schematic diagram of a coil positioning method according to an embodiment of the present application;

FIG. 3 is a schematic diagram of a position at which a radio frequency coil enters the maximum FOV according to an embodiment of this application;

FIG. 4 is a schematic diagram of a position at which a radio frequency coil enters the maximum FOV according to an embodiment of this application;

FIG. 5 is a schematic diagram of a position at which a radio frequency coil enters the maximum FOV according to an embodiment of this application;

FIG. 6 is a schematic diagram of an anatomical distribution relationship according to an embodiment of this application;

FIG. 7 is a schematic diagram of an anatomical distribution relationship according to an embodiment of this application;

FIG. 8 is a schematic diagram of a reference distance determining method according to an embodiment of the present application;

FIG. 9 is a schematic diagram of magnetic field intensity distribution generated by a transceiver coil and a radio frequency coil according to an embodiment of the present application;

FIG. 10 is a schematic diagram of a signal intensity change rate curve according to an embodiment of the present application;

FIG. 11 is a schematic diagram of a signal intensity change rate curve according to an embodiment of the present application;

FIG. 12 is a schematic diagram of a magnetic resonance scanning procedure according to an embodiment of the present application;

FIG. 13 is an example diagram of coil positioning according to an embodiment of the present application; and

FIG. 14 is an example diagram of coil positioning according to an embodiment of the present application.

### DETAILED DESCRIPTION

The aforementioned and other features of the embodiments of the present application will become apparent from the following description with reference to the drawings. In the description and drawings, specific implementations of the present application are disclosed in detail, and part of the implementations in which the principles of the embodiments of the present application may be employed are indicated. It should be understood that the present application is not limited to the described implementations. On the contrary, the embodiments of the present application include all modifications, variations, and equivalents which fall within the scope of the appended claims.

In the embodiments of the present application, the terms “first”, “second”, etc. are used to distinguish different elements from one another by title, but do not represent the spatial arrangement, temporal order, etc. of the elements, and the elements should not be limited by said terms. The term “and/or” includes any one of and all combinations of one or more associated listed terms. The terms “comprise”, “include”, “have”, etc., refer to the presence of stated features, elements, components, or assemblies, but do not exclude the presence or addition of one or more other features, elements, components, or assemblies.

In the embodiments of the present application, the singular forms “a”, “the”, etc. include plural forms, and should be broadly construed as “a type of” or “a class of” rather than being limited to the meaning of “one”. Furthermore, the term “the” should be construed as including both the singular and plural forms, unless otherwise explicitly specified in the context. In addition, the term “according to” should be construed as “at least in part according to . . . ”, and the term “based on” should be construed as “at least in part based on . . . ”, unless otherwise explicitly specified in the context.

The features described and/or illustrated for one embodiment may be used in one or more other embodiments in an identical or similar manner, combined with features in other embodiments, or replace features in other embodiments. The term “include/comprise” when used herein refers to the presence of features, integrated components, steps, or assemblies, but does not exclude the presence or addition of one or more other features, integrated components, steps, or assemblies.

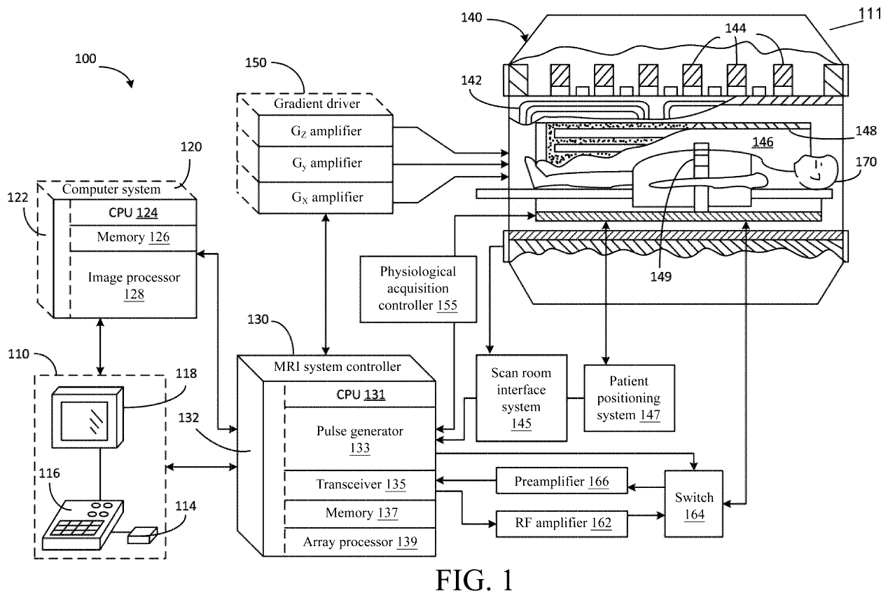

For ease of understanding, FIG. 1 shows a magnetic resonance imaging (MRI) system 100 according to some embodiments of the present invention.

The MRI system 100 includes a scanning unit 111. The scanning unit 111 is used to perform a magnetic resonance scan on a subject (for example, a human body) 170 to generate image data of a region of interest of the subject 170. The region of interest may be a predetermined anatomical site or anatomical tissue. A cold head 61 is disposed on an outer housing of a magnet of the magnetic resonance imaging system. This is only for illustration, and embodiments of the present application are not limited thereto.

Operation of the MRI system 100 is controlled by an operator workstation 110, and the operator workstation 110 includes an input device 114, a control panel 116, and a display 118. The input device 114 may be a joystick, a keyboard, a mouse, a trackball, a touch-activated screen, voice control, or any similar or equivalent input device. The control panel 116 may include a keyboard, a touch-activated screen, voice control, a button, a slider, or any similar or equivalent control device. The operator workstation 110 is coupled to and communicates with a computer system 120, and the computer system enables an operator to control the generation and viewing of an image on the display 118. The computer system 120 includes a plurality of components that communicate with one another by means of an electrical and/or data connection module 122. The connection module 122 may employ a direct wired connection, a fiber optic connection, a wireless communication link, etc. The computer system 120 may include a central processing unit (CPU) 124, a memory 126, and an image processor 128. In some embodiments, the image processor 128 may be replaced with an image processing function implemented in the CPU 124. The computer system 120 may be connected to an archival media device, a persistent or backup memory, or a network. The computer system 120 may be coupled to and communicate with a separate MRI system controller 130.

The MRI system controller 130 includes a set of components that communicate with one another by means of an electrical and/or data connection module 132. The connection module 132 may employ a direct wired connection, a fiber optic connection, a wireless communication link, etc. The MRI system controller 130 may include a CPU 131, a sequential pulse generator 133 that communicates with the operator workstation 110, a transceiver (or an RF transceiver) 135, a memory 137, and an array processor 139. In some embodiments, the sequential pulse generator 133 may be integrated into a resonance assembly 140 of the scanning unit 111 of the MRI system 100. The MRI system controller 130 may receive a command from the operator workstation 110, and is coupled to the scanning unit 111, to indicate an MRI scan sequence that is to be executed during an MRI scan, so as to control the scanning unit 111 to execute the above-described magnetic resonance scan procedure. The MRI system controller 130 is further coupled to and communicates with a gradient driver system 150, and the gradient driver system is coupled to a gradient coil assembly 142 to generate a magnetic field gradient during the MRI scan.

The sequential pulse generator 133 may further receive data from a physiological acquisition controller 155. The physiological acquisition controller receives signals from a plurality of different sensors (for example, electrocardiogram (ECG) signals from electrodes attached to a patient), the sensors being connected to the subject or patient 170 undergoing the MRI scan. The sequential pulse generator 133 is coupled to and communicates with a scan room interface system 145, and the scan room interface system receives signals from various sensors associated with the state of the resonance assembly 140. The scan room interface system 145 is further coupled to and communicates with a patient positioning system 147, and the patient positioning system sends and receives signals to control the movement of a patient table to a desired position to perform the MRI scan.

The MRI system controller 130 provides gradient waveforms to the gradient driver system 150, and the gradient driver system includes Gₓ (x direction), G_{y} (y direction), and G_{z} (z direction) amplifiers, etc. Each of the Gₓ, G_{y}, and G_{z} gradient amplifiers excites a corresponding gradient coil in the gradient coil assembly 142, to generate a magnetic field gradient used to spatially encode an MR signal during an MRI scan. The gradient coil assembly 142 is disposed within the resonance assembly 140. The resonance assembly further includes a superconducting magnet having a superconducting coil 144, and during operation, the superconducting coil provides a static uniform longitudinal magnetic field B₀ (also referred to as a main magnetic field or a static magnetic field) that runs through a cylindrical imaging volume 146. The resonance assembly 140 further includes an RF body coil 148, which, in operation, provides a transverse magnetic field B₁ (also referred to as a radio frequency field), the transverse magnetic field B₁ being substantially perpendicular to B₀ throughout the entire cylindrical imaging volume 146. The resonance assembly 140 may further include an RF surface coil 149, and the RF surface coil is used to image different anatomical structures of the patient undergoing the MRI scan. The RF body coil 148 and the RF surface coil 149 may be configured to operate in a transmit and receive mode, a transmit mode, or a receive mode.

The x direction may also be referred to as a frequency encoding direction or a kₓ direction in k-space. The y direction may be referred to as a phase encoding direction or a k_{y} direction in the k-space. Gₓ can be used for frequency encoding or signal readout, and is generally referred to as a frequency encoding gradient or a readout gradient. G_{y} can be used for phase encoding, and is generally referred to as a phase encoding gradient. G_{z} can be used for slice (layer) position selection to obtain k-space data. It should be noted that a layer selection direction, a phase encoding direction, and a frequency encoding direction may be modified according to actual requirements.

The subject or patient 170 of the MRI scan may be positioned within the cylindrical imaging volume 146 of the resonance assembly 140. The transceiver 135 in the MRI system controller 130 generates RF excitation pulses that are amplified by an RF amplifier 162 and provided to the RF body coil 148 by means of a transmit/receive switch (T/R switch) 164.

As described above, the RF body coil 148 and the RF surface coil 149 may be used to transmit an RF excitation pulse and/or receive obtained MR signals from the patient undergoing the MRI scan. MR signals emitted by excited nuclei in the patient undergoing the MRI scan may be sensed and received by the RF body coil 148 or the RF surface coil 149 and sent back to a pre-amplifier 166 by means of the T/R switch 164. The T/R switch 164 may be controlled by a signal from the sequential pulse generator 133 to electrically connect, when in the transmit mode, the RF amplifier 162 to the RF body coil 148, and to connect, when in the receive mode, the pre-amplifier 166 to the RF body coil 148. The T/R switch 164 may further enable the RF surface coil 149 to be used in the transmit mode or the receive mode.

In some embodiments, the MR signals sensed and received by the RF body coil 148 or the RF surface coil 149 and amplified by the pre-amplifier 166 are stored as a raw k-space data array in the memory 137 for post-processing. A reconstructed magnetic resonance image may be obtained by transforming/processing the stored raw k-space data.

In some embodiments, the MR signals sensed and received by the RF body coil 148 or the RF surface coil 149 and amplified by the pre-amplifier 166 are demodulated, filtered, and digitized in a receiving portion of the transceiver 135, and transmitted to the memory 137 in the MRI system controller 130. For each image that is to be reconstructed, the data is rearranged into a separate k-space data array, each of the separate k-space data arrays is inputted into the array processor 139, and the array processor is operated to transform the data into an array of image data by means of a Fourier transform.

The array processor 139 uses a transform method, most commonly a Fourier transform, to create images from the received MR signals. These images are transmitted to the computer system 120 and stored in the memory 126. In response to commands received from the operator workstation 110, the image data may be stored in a long-term memory, or may be further processed by the image processor 128 and transmitted to the operator workstation 110 for presentation on the display 118.

In various embodiments, components of the computer system 120 and the MRI system controller 130 may be implemented on the same computer system or on a plurality of computer systems. It should be understood that the MRI system 100 shown in FIG. 10 is intended for illustration. A suitable MRI system may include more, fewer, and/or different components.

The MRI system controller 130 and the image processor 128 may separately or collectively include a computer processor and a storage medium. The storage medium records a predetermined data processing program that is to be executed by the computer processor. For example, the storage medium may store a program used to implement scanning (for example, a scan procedure and an imaging sequence), image reconstruction, image processing, etc. For example, the storage medium may store a program used to implement the magnetic resonance imaging method according to the embodiments of the present invention. The above storage medium may include, for example, a ROM, a floppy disk, a hard disk, an optical disk, a magneto-optical disk, a CD-ROM, or a non-volatile memory card.

The aforementioned “imaging sequence” (also referred to below as a scan sequence or a pulse sequence) is a combination of pulses that have specific amplitudes, widths, directions, and time sequences, and that are applied when a magnetic resonance imaging scan is executed. These pulses typically may include, for example, a radio-frequency pulse and a gradient pulse. The radio-frequency pulses may include, for example, radio-frequency excitation pulses, radio-frequency refocusing pulses, inverse recovery pulses, etc. The gradient pulses may include, for example, the aforementioned gradient pulse used for layer selection, gradient pulse used for phase encoding, gradient pulse used for frequency encoding, gradient pulse used for phase shifting (phase shift), gradient pulse used for dispersion of phases (dephasing), etc.

Typically, a plurality of scan sequences can be preset in the magnetic resonance system, so that a sequence suitable for clinical examination requirements can be selected. The clinical examination requirements may include, for example, an imaging site, an imaging function, an imaging effect, and the like.

Description is made below in conjunction with the embodiments.

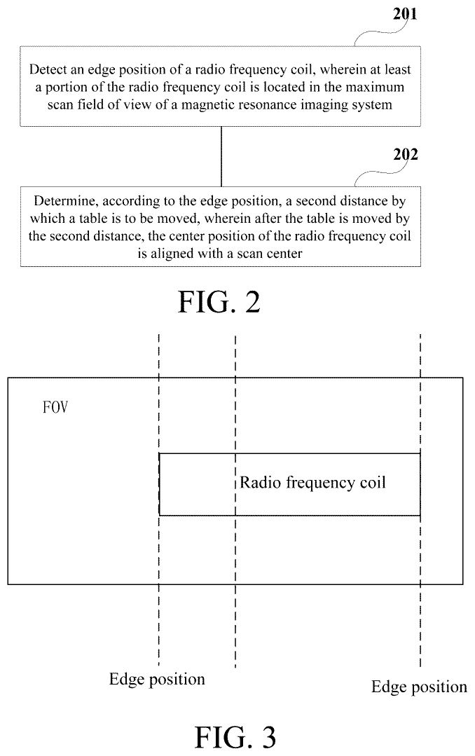

Embodiments of the present application provide a coil positioning method for a magnetic resonance imaging system, and the purpose of the embodiments of the present application is to position the center position of a coil, for example, to a scan center. FIG. 2 is a schematic diagram of a coil positioning method for a magnetic resonance imaging system according to an embodiment of the present application.

As shown in FIG. 2, the process includes at step 201 detecting an edge position of a radio frequency coil, wherein at least a portion of the radio frequency coil is located in the maximum scan field of view of a magnetic resonance imaging system, and at step 202 determining, according to the edge position, a second distance by which a table is to be moved, wherein after the table is moved by the second distance, the center position of the radio frequency coil is aligned with a scan center.

The above “aligned” may include the case in which the center position of the radio frequency coil is aligned with the scan center in both the superior/inferior (SI) direction and the right/left (RL) direction, or may include alignment only in the SI direction. The scan center refers to the center of the main magnetic field of the magnetic resonance imaging system, or another physical position defined in the magnetic field space (or scanning bore).

Coil positioning in the following example refers to taking positioning in the table moving direction (z direction) as an example. For example, a first distance, a second distance, and various involved positions all refer to distances or positions in the table moving direction (z direction). The first distance and the second distance by which the table is moved are distance vectors, that is, the distance and the moving direction are included. When the table is outside the scanning cavity, the side close to the scanning bore is called the table head, and the side away from the scanning bore is called the table tail. The detailed description is provided below.

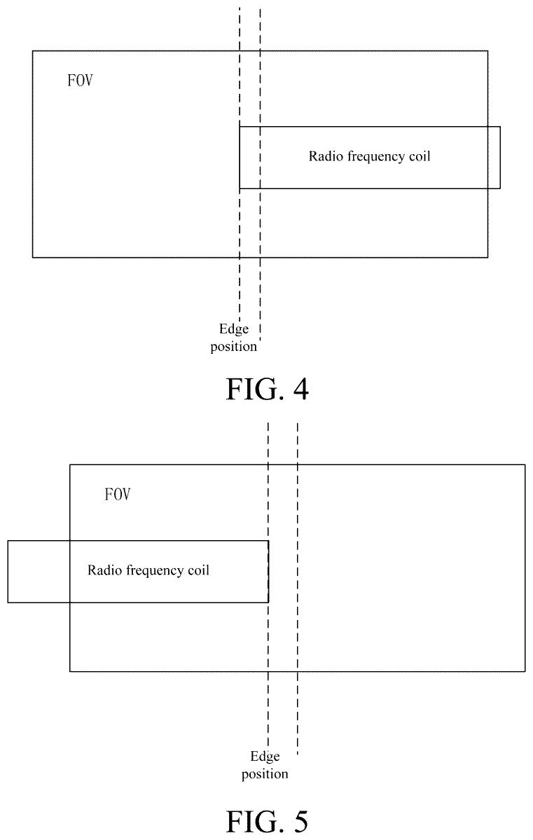

In some embodiments, in 201, the edge position of the radio frequency coil is detected when at least a portion of the radio frequency coil is located in the maximum scan field of view of the magnetic resonance imaging system. The maximum field of view (FOV) refers to the maximum spatial range of an imaging region that can be captured by the magnetic resonance imaging system during one time of imaging, and the FOV is a two-dimensional or three-dimensional spatial range. Taking a two-dimensional spatial range in the superior-inferior direction and the left-right direction as an example, for example, the maximum FOV may be set to 480 mm×480 mm, which is only an example herein and the embodiments of the present application are not limited thereto. FIG. 3 to FIG. 5 are schematic diagrams of a position at which a radio frequency coil enters the maximum FOV according to an embodiment of this application, wherein as shown in FIG. 3, the radio frequency coil completely enters the FOV, and as shown in FIG. 4 and FIG. 5, a portion of the radio frequency coil is located within the maximum FOV.

In some embodiments, in order to make at least a portion of the radio frequency coil enter the maximum scan field of view of the magnetic resonance imaging system, before 201, the method may further include: determining a first distance by which the table is to be moved in the magnetic resonance imaging system, wherein after the table is moved by the first distance, at least a portion of the radio frequency coil enters the maximum scan field of view.

In some embodiments, an operator may determine the first distance based on experience, and after manually adjusting the table to move by the first distance, the operator may determine through observation that at least a portion of the radio frequency coil enters the maximum scan field of view.

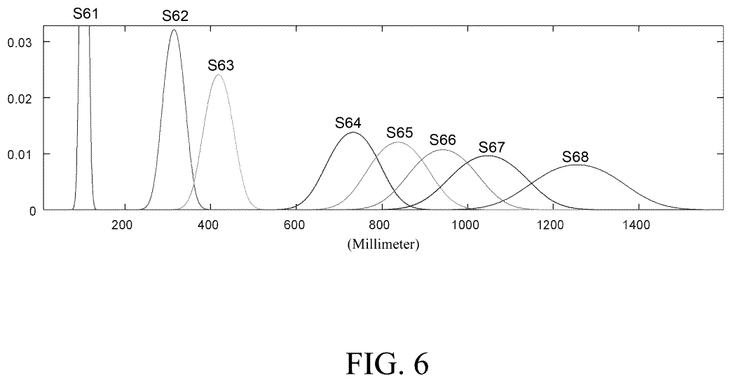

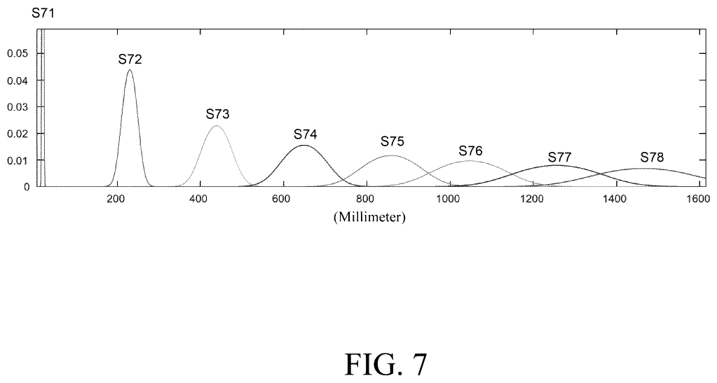

In some embodiments, the first distance may be determined according to a site to be examined and an anatomical distribution relationship. The anatomical distribution relationship is obtained in advance based on experience. FIG. 6 and FIG. 7 are schematic diagrams of an anatomical distribution relationship according to an embodiment of this application. FIG. 6 and FIG. 7 show distribution of position coordinates of various anatomical positions of a human body in the z direction, or distribution of distances between various anatomical positions of the human body and the head or the sole in the z direction, under the average height. As shown in FIG. 6, when the human body is first placed on the table, the head is closer to the table head, that is, the head top direction is used as the coordinate origin, and anatomical sites represented by S61 to S68 are respectively: head or neck, shoulder, chest, abdomen, pelvis, buttock, long bone, and knee. As shown in FIG. 7, when the human body is first placed on the table, the foot is closer to the table head, that is, the sole direction is used as the coordinate origin, and anatomical sites represented by S71 to S78 are respectively: foot or ankle, leg, knee, long bone, buttock, pelvis, abdomen, and chest. According to FIG. 6 and FIG. 7, the range of the position coordinates of each anatomical position of the human body in the z direction, or the range of the distance between each anatomical position of the human body and the head or the sole in the z direction, may be determined.

In some embodiments, a relationship table between each anatomical site and a reference distance may be pre-created according to the anatomical distribution relationship. The reference distance is a distance by which the table needs to be moved to ensure that at least a portion or all of the anatomical site is positioned within the maximum scan field of view within the scanning bore of the magnetic resonance imaging system. During scanning, since the human body and a local radio frequency coil coupled to the anatomical site (for example, by sleeving, covering, etc.) are both located on the table, the distance by which the table needs to be moved means the distance by which the anatomical site needs to be moved, and also means the distance by which the local radio frequency coil coupled to the anatomical site needs to be moved, and the above expressions can be interchanged.

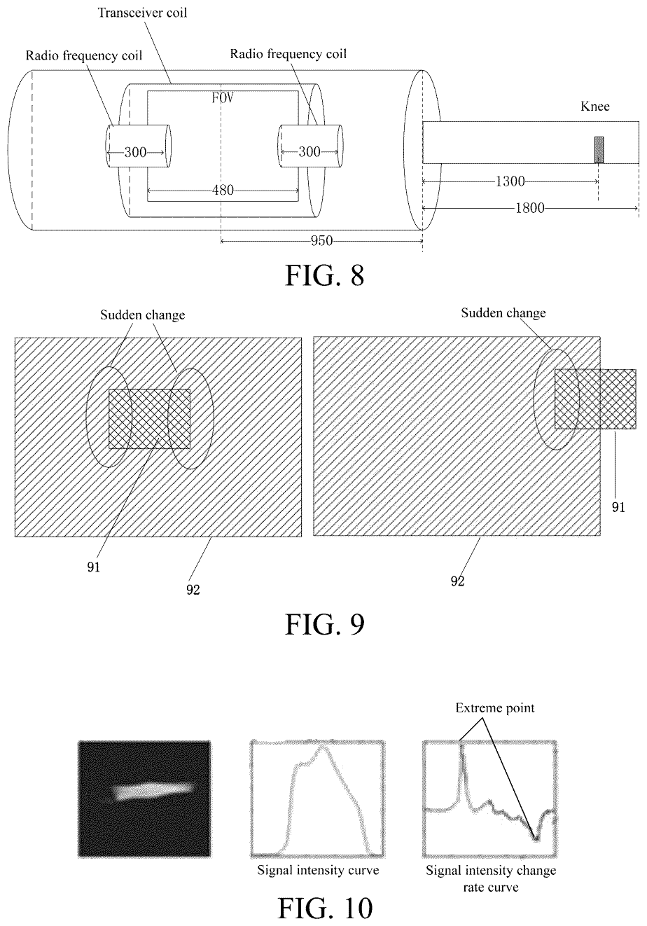

FIG. 8 is a schematic diagram of a reference distance determining method according to an embodiment of the present application. As shown in FIG. 8, it is assumed that the maximum scan field of view (FOV) is 480 mm in the z direction, the distance between the scan center and the edge of the scanning bore is 950 mm, and the reference distance is equal to 950+x, wherein x is the distance between each anatomical position determined according to FIG. 6 or FIG. 7 and the head or the sole in the z direction. Taking the knee in FIG. 6 as an example, the distance of the knee relative to the head is 1300 mm, and the reference distance corresponding to the knee is 1300+950=2250 mm, that is, the distance by which the table needs to be moved when at least a portion or all of the knee is positioned within the maximum scan field of view in the scanning bore of the magnetic resonance imaging system. Table 1 is a schematic table of a relationship between an anatomical site and a reference distance according to an embodiment of this application.

TABLE 1
(average height)

| | Top of head as the | Soles as the |
|---|---|---|
| Site to | origin-reference | origin-reference |
| be examined | distance (mm) | distance (mm) |
| Knee joint | 2250 | 1600 |
| Chest | 1400 | 2450 |
| . . . | . . . | . . . |
| Abdomen | 1650 | 2250 |
| Pelvic cavity | 1850 | 2050 |

In some embodiments, optionally, the height or the gender of a scanned subject may also be considered, and respective relationship tables are created for different height ranges or genders. As shown in Table 2 and Table 3, relationship tables are respectively created for different height ranges. Only two tables are used as examples herein, the embodiments of the present application are not limited thereto, and the examples are not described herein one by one.

TABLE 2
(height 180 cm or more)

| | Top of head as the | Soles as the |
|---|---|---|
| Site to | origin-reference | origin-reference |
| be examined | distance (mm) | distance (mm) |
| Knee joint | 2350 | 1700 |
| Chest | 1500 | 2550 |
| . . . | . . . | . . . |
| Abdomen | 1750 | 2350 |
| Pelvic cavity | 1950 | 2150 |

TABLE 3
(height 160 cm or less)

| | Top of head as the | Soles as the |
|---|---|---|
| Site to | origin-reference | origin-reference |
| be examined | distance (mm) | distance (mm) |
| Knee joint | 2150 | 1500 |
| Chest | 1300 | 2350 |
| . . . | . . . | . . . |
| Abdomen | 1550 | 2150 |
| Pelvic cavity | 1750 | 1950 |

In some embodiments, the first distance may be determined according to the relationship table and the site to be examined, or according to the relationship table and the site to be examined as well as the height or the gender of the scanned subject, that is, the reference distance corresponding to the site to be examined in the table is used as the first distance. For example, when the knee joint needs to be scanned, the system first receives input information of the site to be examined by means of a user interface. According to the relationship table 1, it may be determined that the reference distance corresponding to the knee joint is 2250 mm, and the reference distance is used as the first distance. In addition, the reference distance may be dynamically adjusted according to the height information of the scanned subject. For example, for a patient with a greater height, the reference distance corresponding to the knee joint may be determined with reference to Table 2 or Table 3 or calculated according to a dynamic algorithm, so as to determine the first distance, and details are not described herein.

In some embodiments, after the first distance is determined, a button may be provided at the table side, or table movement may be triggered by means of the system user interface. For example, the first distance may be recorded in advance in a scan protocol corresponding to the site to be examined, and the table may be controlled to move by the first distance upon pressing the button (at this time, both the human body and the coil are located on the table). After the table is moved by the first distance, at least a portion of the radio frequency coil coupled to the site to be examined of the human body enters the maximum scan field of view.

By means of this method, the determining of the first distance is based on both an anatomical model and empirical data, and individualized anatomical differences of the patient can be considered, which improves positioning accuracy while improving automatic positioning efficiency, providing a basis for subsequent fine positioning.

In some embodiments, after at least a portion of the radio frequency coil enters the maximum scan field of view of the magnetic resonance imaging system, the method further includes: executing positioning scanning and acquiring a magnetic resonance signal based on the radio frequency coil, and detecting the edge position according to the magnetic resonance signal in 201. A person skilled in the art understands that positioning scanning is also referred to as three-plane positioning scanning, which is executed before formal diagnostic scanning. At least one of a coronal scout image, a sagittal scout image, and an axial scout image of the subject may be acquired based on the positioning scanning. Based on this scout image, scan parameters, such as a scan range, a scan sequence, radio frequency power/a radio frequency transmit gain, etc., of a formal scan, may be determined. When the positioning scanning is executed, in this embodiment of this application, a scan sequence that is preset by the system and that is used for positioning scanning may be transmitted. Positioning scanning is executed by using a transceiver coil and the radio frequency coil. The transceiver coil includes a body coil. The radio frequency coil includes a wireless coupling coil, for example, at least one of a head coil, a knee coil, an ankle joint coil, an abdomen coil, an elbow coil, a chest coil, a spine coil, a neck coil, and a shoulder coil. The radio frequency coil is configured to cooperate with the site to be examined and is coupled to the magnetic resonance system in a wireless cable connection manner. For example, the radio frequency coil may be configured to wrap/surround/cover/be close to the site to be examined. The radio frequency coil is configured to be coupled to a transceiver coil (for example, a body coil 148 in FIG. 1) of the magnetic resonance system to receive a radio frequency pulse transmitted from the transceiver coil, so as to generate a radio frequency field exciting the scanned subject. The radio frequency coil also transmits the magnetic resonance signal received from the scanned subject to the transceiver coil, so as to complete acquisition of the magnetic resonance signal.

In some embodiments, optionally, the transceiver coil and the radio frequency coil may have the same electrical or electromagnetic structure, such as a birdcage-like structure. However, the embodiments of the present application are not limited thereto. When the transceiver coil has another electrical structure, the radio frequency coil may also correspondingly include a similar electrical structure. Therefore, by using the electromagnetic principle, the transceiver coil and the radio frequency coil generate electromagnetic coupling (mutual inductance) in a wireless manner to transmit and receive radio frequency signals. That is, during scanning, the transceiver coil and the radio frequency coil are close to each other to generate electromagnetic coupling, and the coupling is strong. Therefore, when the transceiver coil transmits a radio frequency pulse, a current is induced in the radio frequency coil, that is, the radio frequency pulse is transmitted from the transceiver coil to the radio frequency coil by using an induced magnetic field, and the radio frequency pulse transmitted to the radio frequency coil generates a uniform magnetic field B1. The excitation subject to be examined generates resonance to generate a transverse magnetization vector, and generates a magnetic resonance signal that can be acquired, wherein the magnetic resonance signal is sensed and received by the radio frequency coil. When the radio frequency coil receives the magnetic resonance signal, a current is induced in the transceiver coil, that is, the magnetic resonance signal is transmitted from the radio frequency coil to the transceiver coil by using an induced magnetic field, and is transmitted to a receiver chain module of the system by using a transmission cable connected to the transceiver coil.

Therefore, in 201, the edge position of the radio frequency coil on the table in the maximum scan field of view may be detected according to the acquired magnetic resonance signal. For ease of understanding, the principle that the edge position of the radio frequency coil can be detected according to the acquired magnetic resonance signal will be described below.

FIG. 9 is a schematic diagram of distribution of magnetic field intensity generated by a transceiver coil and a radio frequency coil according to an embodiment of the present application. As shown in FIG. 9, the inventor finds by means of simulation that when a coupling coil 91 as a radio frequency coil is in a transceiver coil 92, magnetic field intensity induced inside the radio frequency coil 91 is uniform, and this characteristic does not change with different positions of the coupling coil 91 in the transceiver coil 92. When the coupling coil 91 is completely or partially located in the magnetic field region of the transceiver coil 92, the magnetic field intensity induced inside the radio frequency coil 91 is uniform even if it is not completely located at the center of the transceiver coil 92. However, the magnetic field intensity outside the coupling coil 91 is completely different from the magnetic field intensity inside the coupling coil 91, and since the B1 magnetic field inside the coupling coil is larger, the magnetic field on both sides of the edge position of the coupling coil 91 has a sudden change. Therefore, as long as the coupling coil is moved within the FOV, the position at which the signal intensity changes the most can be identified by using the captured magnetic resonance signal, and this position is the edge position of the coupling coil.

In some embodiments, in 201, a signal intensity curve projected in the moving direction of the table is calculated according to the magnetic resonance signal, that is, a signal intensity curve projected in the z direction is obtained, and the curve reflects signal intensity at different positions in the z direction. Then, a position with the greatest signal intensity change in the signal intensity curve is identified as the edge position. A signal intensity change difference between adjacent positions may be calculated point by point according to the signal intensity curve. The position with the largest difference is taken as the edge position, that is, a sudden change point of the signal intensity from low to high or from high to low reflects the edge position of the coil. For example, a differential operation (for example, using a diff function) may be performed on the signal intensity curve to obtain a signal intensity change rate curve (or referred to as a signal intensity change gradient curve), and the coordinates of one or two extreme points (signal sudden change points) of the signal intensity change rate curve are the coordinates of the edge position of the radio frequency coil.

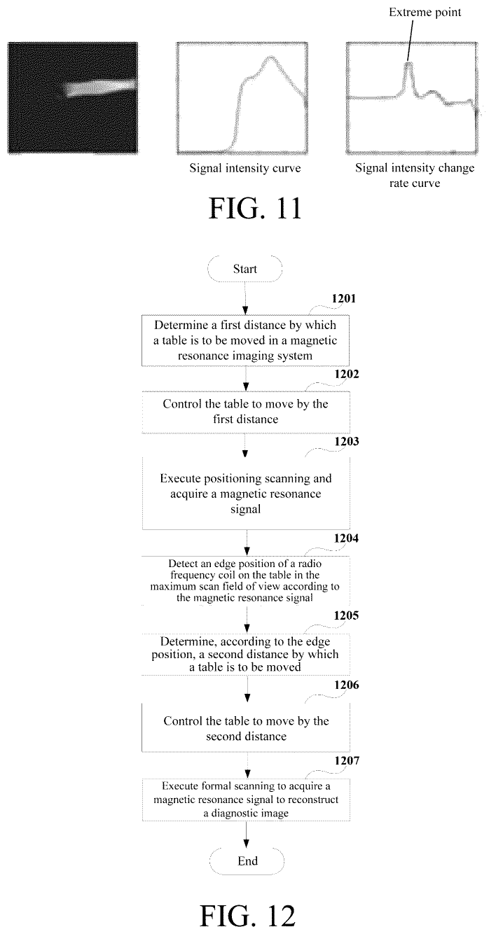

In some embodiments, for the case shown in FIG. 3, the coupling coil completely enters the FOV, and the signal intensity change rate curve that can be obtained is shown in FIG. 10. As shown in FIG. 10, the signal intensity change rate curve has two extreme points, which correspond to the coordinates of two left and right edge positions in the z direction of the coupling coil. In the case shown in FIG. 4, only the left portion of the coupling coil enters the FOV, and the signal intensity change rate curve that can be obtained is shown in FIG. 11. As shown in FIG. 11, the signal intensity change rate curve has one extreme point, which corresponds to the coordinates of the left edge position of the coupling coil in the z direction. In the case shown in FIG. 5, one extreme point may also be obtained, and examples are not provided herein. In FIG. 10 and FIG. 11, an example in which the edge position of the FOV is used as the coordinate 0 point is used, and this embodiment of this application is not limited thereto.

It should be noted that the coordinates of the edge position obtained according to the above signal intensity change rate curve include coordinates on the coordinate axis of the moving direction of the table and coordinates on the coordinate axis of the width direction of the table. The coordinates of the edge position to be used when calculating the second distance later refer to the coordinates on the coordinate axis of the moving direction of the table. The origin of the coordinate axis may be set as the center point of the FOV or an edge point of the FOV, and the embodiments of the present application are not limited thereto.

In some embodiments, in 202, the second distance by which the table is to be moved may be determined according to the one or two edge positions. When the obtained second distance is a positive number, it indicates that the table needs to be moved in the entry direction of the table by the absolute value of the second distance, and when the obtained second distance is a negative number, it indicates that the table needs to be moved in the exit direction of the table by the absolute value of the second distance. Vice versa, the embodiments of the present application are not limited thereto, and the distance is specifically related to the stipulation of the positive and negative directions of the coordinate axis. After the table is moved by the second distance again, the center of the radio frequency coil may be aligned with the scan center, positioning of the coil is completed, and formal diagnostic scanning is started.

In some embodiments, the center position of the radio frequency coil may be calculated according to one edge position and the size of the radio frequency coil. The length L of the radio frequency coil in the z direction is known. Therefore, assuming that the scan center is the origin, the negative axis is close to the table head, and the positive axis is close to the table tail, when the edge position used is the edge position close to the table head, the coordinates of the center position of the radio frequency coil are equal to the coordinates of the edge position plus L/2, and when the edge position used is the edge position close to the table tail, the coordinates of the center position of the radio frequency coil are equal to the coordinates of the edge position minus L/2. In the case that the positive axis is close to the table head, and the negative axis is close to the table tail, when the edge position used is the edge position close to the table head, the coordinates of the center position of the radio frequency coil are equal to the coordinates of the edge position minus L/2, and when the edge position used is the edge position close to the table tail, the coordinates of the center position of the radio frequency coil are equal to the coordinates of the edge position plus L/2. Then, the distance (vector) between the center position and the scan center is calculated as the second distance. Since it is assumed that the z-direction coordinates of the scan center are 0, the absolute value of the coordinates of the center position may be used as the second distance for movement, and whether the movement direction is the table entry direction or the table exit direction is determined according to the positive and negative of the coordinates of the center position. The above example takes the scan center as the coordinate origin, but this embodiment of the present application is not limited thereto, for example, the edge position of the FOV may also be used as the coordinate origin, which is not illustrated one by one herein.

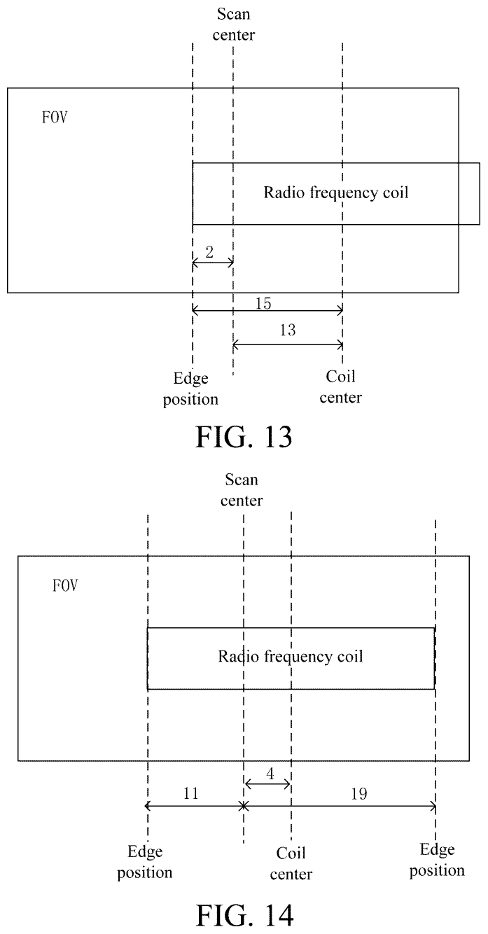

FIG. 13 is an example diagram of coil positioning according to an embodiment of the present application. As shown in FIG. 13, the left portion of the radio frequency coil is located within the FOV, the detected edge position is 2 cm away from the scan center, and half of the length of the coil in the z direction is 15 cm. Therefore, it is determined that the second distance is 13 cm, that is, after the table needs to be moved by 13 cm again in the table entry direction, the center of the radio frequency coil is aligned with the scan center.

FIG. 14 is an example diagram of coil positioning according to an embodiment of the present application. As shown in FIG. 14, the radio frequency coil is completely located within the FOV, two detected edge positions are respectively 11 cm (the actual sign is negative) and 19 cm away from the scan center, and the second distance is determined by using one of the edge positions and the size of the radio frequency coil. For example, the absolute value of the second distance is 19−15=4 cm or 15−11=4 cm, that is, after the table needs to be moved by 4 cm again in the table entry direction, the center of the radio frequency coil is aligned with the scan center.

In some embodiments, the center position of the radio frequency coil may be calculated according to two edge positions; and the distance between the center position and the scan center may be calculated as the second distance. In the case that two edge positions are obtained, the size of the radio frequency coil is not required, and the average value of the coordinates of the two edge positions is calculated as the center position of the radio frequency coil. Then, the distance (vector) between the center position and the scan center is calculated as the second distance. Since the z-direction coordinates of the scan center are 0, the absolute value of the coordinates of the center position may be used as the second distance for movement, and whether the movement direction is the table entry direction or the table exit direction is determined according to the positive and negative of the coordinates of the center position.

As shown in FIG. 14, the radio frequency coil is completely located within the FOV, and detected two edge positions are respectively 11 cm (the actual sign is negative) and 19 cm away from the scan center. Therefore, it is determined that the absolute value of the second distance is (19−11)/2=4 cm, that is, after the table needs to be moved by 4 cm again in the table entry direction, the center of the radio frequency coil is aligned with the scan center.

FIG. 12 is a schematic diagram of a magnetic resonance scanning procedure. As shown in FIG. 12, the process includes at step 1201 determining a first distance by which a table is to be moved in a magnetic resonance imaging system, at step 1202 controlling the table to move by the first distance, at step 1203 executing positioning scanning and acquiring a magnetic resonance signal, at step 1204 detecting an edge position of a radio frequency coil on the table in the maximum scan field of view according to the magnetic resonance signal, at step 1205 determining, according to the edge position, a second distance by which the table is to be moved, at step 1206 controlling the table to move by the second distance, and at step 1207 executing formal scanning to acquire a magnetic resonance signal to reconstruct a diagnostic image.

Implementations of steps 1201-1207 are as described previously, and will not be repeated herein. In the above scanning procedure, there are two steps of coarse positioning and fine positioning. In coarse positioning, the first distance by which the table is to be moved is determined first, and after the table is moved by the first distance, at least a portion of the radio frequency coil on the table enters the maximum scan field of view in a scanning bore of the magnetic resonance imaging system. In fine positioning, the second distance by which the table is to be moved is determined, and after the table is moved by the second distance again, the center position of the radio frequency coil on the table is aligned with the scan center. Thus, positioning of the radio frequency coil is completed.

It should be noted that the above figures merely schematically illustrate the embodiments of the present application, but the present application is not limited thereto. For example, the order of execution between operations may be appropriately adjusted. In addition, some other operations may be added or some operations may be omitted. Those skilled in the art can make appropriate variations according to the above content, rather than being limited by the disclosure of the foregoing accompanying drawings.

The above embodiments merely provide illustrative descriptions of the embodiments of the present application. However, the present application is not limited thereto, and suitable variations may be made on the basis of the above embodiments. For example, each of the above embodiments may be used independently, or one or more of the above embodiments may be combined.

By means of the above embodiment, a second distance by which a table is to be moved is determined by detecting an edge position of a radio frequency coil when at least a portion of the radio frequency coil is located in the maximum scan field of view of a magnetic resonance imaging system. Therefore, automatic positioning of the radio frequency coil can be achieved, and no human intervention is required, which can simplify the scanning procedure and save scanning time. In addition, since it is no longer necessary to set a laser light for positioning, hardware costs can be saved, and hazards caused by the laser light irradiating the eyes during head scanning can also be prevented.

Embodiments of the present application further provide a magnetic resonance imaging system. The magnetic resonance imaging system includes a table, a transceiver coil, a radio frequency coil, a scanning unit, and a controller. Another configuration of the magnetic resonance imaging system may be shown in FIG. 1, and what are the same will not be repeated herein. For an implementation thereof, reference may be made to the aforementioned embodiments, which will not be repeated herein.

In some embodiments, what differs from the foregoing magnetic resonance imaging system in FIG. 1 is that the controller 130 is configured to execute the foregoing coil positioning method.

In some embodiments, the controller 130 (which may also be a processor) comprises a computer processor and a storage medium. The storage medium has recorded thereon a predetermined data processing program to be executed by the computer processor. For example, the storage medium may store a program configured to implement scanning processing (for example, including waveform design/conversion, and the like), image reconstruction, image processing, etc. For example, the storage medium may store a coil positioning method used to implement this embodiment of the present application. For example, the controller determines a first distance by which the table is to be moved in the magnetic resonance imaging system, and controls the table to move by the first distance. The controller controls a sequential pulse generator to generate a positioning sequence, executes positioning scanning, and controls a coil to acquire a magnetic resonance signal. The controller detects an edge position of the radio frequency coil on the table in the maximum scan field of view according to the magnetic resonance signal, and determines the second distance by which the table is to be moved according to the edge position. The controller controls the table to move by the second distance. For a specific implementation, reference may be made to the foregoing embodiments, which will not be repeated herein.

The above storage medium may include, for example, a ROM, a floppy disk, a hard disk, an optical disk, a magneto-optical disk, a CD-ROM, or a non-volatile memory card.

The embodiments of the present application further provide a computer-readable program. When the program is executed in an apparatus or an MRI system, the program enables a computer to execute, in the apparatus or the MRI system, the method according to the foregoing embodiments.

Embodiments of the present application further provide a storage medium having a computer-readable program stored thereon. The computer-readable program causes a computer to execute the method according to the foregoing embodiments in an apparatus or MRI system.

The above apparatus and method of the present application can be implemented by hardware, or can be implemented by hardware in combination with software. The present application relates to such a computer-readable program that when executed by a logic component, the program causes the logic component to implement the foregoing apparatus or a constituent component, or causes the logic component to implement various methods or steps as described above. The present application further relates to a storage medium for storing the above program, such as a hard disk, a magnetic disk, an optical disc, a DVD, a flash memory, etc.

The method/apparatus described with reference to the embodiments of the present application may be directly embodied as hardware, a software module executed by a processor, or a combination of the two. For example, one or more of the functional block diagrams and/or one or more combinations of the functional block diagrams shown in the figures may correspond to either software modules or hardware modules of a computer program flow. The foregoing software modules may respectively correspond to the steps shown in the figures. The foregoing hardware modules may be implemented, for example, by consolidating the foregoing software modules by using a field-programmable gate array (FPGA).

The software modules may be located in a RAM memory, a flash memory, a ROM memory, an EPROM memory, an EEPROM memory, a register, a hard disk, a removable disk, a CD-ROM, or any storage medium in other forms known in the art. The storage medium may be coupled to a processor, so that the processor can read information from the storage medium and can write information into the storage medium. Alternatively, the storage medium may be a constituent component of the processor. The processor and the storage medium may be located in an ASIC. The software module may be stored in a memory of a mobile terminal, and may also be stored in a memory card that can be inserted into a mobile terminal. For example, if a device (such as a mobile terminal) uses a large-capacity MEGA-SIM card or a large-capacity flash memory apparatus, then the software modules may be stored in the MEGA-SIM card or the large-capacity flash memory apparatus.

One or more of the functional blocks and/or one or more combinations of the functional blocks shown in the drawings may be implemented as a general-purpose processor, a digital signal processor (DSP), an application-specific integrated circuit (ASIC), a field-programmable gate array (FPGA) or other programmable logic devices, a discrete gate or transistor logic device, a discrete hardware assembly, or any appropriate combination thereof, which is used for executing the functions described in the present application. The one or more functional blocks and/or the one or more combinations of the functional blocks shown in the drawings may alternatively be implemented as a combination of computing devices, such as a combination of a DSP and a microprocessor, a plurality of microprocessors, one or more microprocessors in communication combination with a DSP, or any other such configuration.

The present application is described above with reference to specific implementations. However, it should be clear to those skilled in the art that the foregoing description is merely illustrative and is not intended to limit the scope of protection of the present application. Various variations and modifications may be made by those skilled in the art according to the principle of the present application, and said variations and modifications also fall within the scope of the present application.

1. A coil positioning method for a magnetic resonance imaging system, characterized by comprising:
detecting an edge position of a radio frequency coil, wherein at least a portion of the radio frequency coil is located in the maximum scan field of view of a magnetic resonance imaging system; and
determining, according to the edge position, a second distance by which a table is to be moved, wherein after the table is moved by the second distance, the center position of the radio frequency coil is aligned with a scan center.

2. The method according to claim 1, wherein before detecting the edge position, the method further comprises:
determining a first distance by which the table is to be moved in the magnetic resonance imaging system, wherein after the table is moved by the first distance, at least a portion of the radio frequency coil enters the maximum scan field of view.

3. The method according to claim 2, wherein determining a first distance by which the table is to be moved in the magnetic resonance imaging system comprises:
determining the first distance according to a site to be examined and an anatomical distribution relationship.

4. The method according to claim 1, further comprising:
executing a positioning scan and acquiring a magnetic resonance signal based on the radio frequency coil;
and detecting the edge position according to the magnetic resonance signal.

5. The method according to claim 1, wherein the radio frequency coil comprises a wireless coupling coil.

6. The method according to claim 4, wherein executing a positioning scan and acquiring a magnetic resonance signal comprises:
receiving, via the radio frequency coil, a radio frequency pulse transmitted from a transceiver coil to generate a radio frequency field that excites a scanned subject; and
sending a magnetic resonance signal received from the scanned subject to the transceiver coil via the radio frequency coil, wherein the radio frequency coil and the transceiver coil are electromagnetically coupled.

7. The method according to claim 6, wherein the transceiver coil comprises a body coil.

8. The method according to claim 5, wherein the radio frequency coil comprises at least one of a head coil, a knee coil, an ankle joint coil, an abdomen coil, an elbow coil, a chest coil, a spine coil, a neck coil, and a shoulder coil.

9. The method according to claim 6, wherein detecting the edge position according to the magnetic resonance signal comprises:
calculating a signal intensity curve projected in the moving direction of the table according to the magnetic resonance signal; and
identifying a position with the greatest signal intensity change in the signal intensity curve as the edge position.

10. The method according to claim 9, wherein identifying a position with the greatest signal intensity change in the signal intensity curve as the edge position of the radio frequency coil comprises:
calculating a signal intensity change difference between adjacent positions according to the signal intensity curve; and
determining a position with the greatest difference as the edge position.

11. The method according to claim 1, wherein determining a second distance by which a table is to be moved according to the edge position comprises:
calculating the center position of the radio frequency coil according to one edge position and the size of the radio frequency coil; and
calculating the distance between the center position and the scan center as the second distance.

12. The method according to claim 1, wherein the edge position comprises at least coordinates on a coordinate axis in the moving direction of the table.

13. The method according to claim 1, wherein determining a second distance by which a table is to be moved according to the edge position comprises:
calculating the center position of the radio frequency coil according to two edge positions; and
calculating the distance between the center position and the scan center as the second distance.

14. A magnetic resonance imaging system comprising:
a table;
a transceiver coil;
a radio frequency coil;
a scanning unit; and
a controller, which is configured to execute the coil positioning method according to claim 1.
